(19) United States
(12) Patent Application Publication
(10) Pub. No.: US 2022/0257485 A1
(43) Pub. Date: Aug. 18, 2022

(54) PREBIOTIC COSMETIC COMPOSITIONS AND METHODS FOR THE PREPARATION THEREOF

(71) Applicant: Fanny Coste, London (GB)

(72) Inventor: Fanny Coste, London (GB)

(21) Appl. No.: 17/624,797

(22) PCT Filed: Jul. 2, 2020
(86) PCT No.: PCT/US20/40605

§ 371 Date: Jan. 4, 2022

Related U.S. Application Data
(60) Provisional application No. 62/870,948, filed on Jul. 5, 2019.

(51) Int. Cl. A61K 8/37; A61K 8/365; A61K 8/34; A61Q 19/10

(52) U.S. Cl. CPC A61K 8/375; A61Q 19/10; A61K 8/345; A61K 8/365

(57) ABSTRACT

The present disclosure relates to topical prebiotic cosmetic compositions and methods for their preparation. Benefits of the compositions disclosed herein can include a health benefit of promoting a favorable balance of microorganisms in the body's integumentary system.

### TECHNICAL FIELD

The present disclosure relates to topical prebiotic cosmetic compositions and methods for their preparation. Benefits of the compositions disclosed herein can include a health benefit of promoting a favorable balance of microorganisms in the body's integumentary system, including the skin, cavities, and appendages thereof.

### BACKGROUND

The microbiome is generally considered the collective genomes of the microbes (composed of bacteria, bacteriophage, fungi, protozoa and viruses) that live inside and on the human body. It is believed that we have about 10 times as many microbial cells as human cells.

In the last decades, the microbiome has been negatively impacted by poor dietary habits; overuse of personal care products, disinfectants, antibiotics, and other germ killers; declining contact with vital environmental microbes, including those hosted by wildlife and livestock; and the rise in cesarean section births, which do not expose babies to the valuable bacteria found in the birth canal.

An imbalance of the microbiome, called dysbiosis, can lead to a variety of skin conditions including acne, eczema, rosacea and even premature ageing. While Americans have never been so clean, those conditions have risen dramatically. For instance, the incidence of atopic dermatitis, also known as eczema, has increased 2- to 3-fold in industrialized nations, impacting approximately 15% to 20% of children and 1% to 3% of adults worldwide.

It was not until recently that scientists developed tools allowing them to better tally microbial populations and study how their presence or absence affects human health. The advent of improved and cheaper technology such as next-generation sequencing (NGS) combined with breakthroughs in proteomics and metabolomics have allowed initiatives such as the Human Microbiome Project to identify and quantify thousands of previously unknown microorganisms, totally overlooked by conventional culture methods.

Until recently, unaware of the diversity and role of the skin microbiome, the personal care industry followed a binary approach. Either products were designed to eradicate skin bacteria, targeting those involved in skin disorders but in reality, disturbing the commensal flora too, or they ignored the skin microbiota. Deodorants were the first products developed to target skin microorganisms, with early studies on the effect of topical antibacterial agents on the axilla in 1963. The most common antibacterial ingredients used in deodorants worldwide are farnesol (24%) and triclosan (8%), but because of major safety concerns, they have gradually been restricted or banned in most countries.

There remains a need in the art for cosmetic compositions that not only effectively control undesirable microbial activity but also foster a beneficial microbial balance of the body's integumentary system.

### SUMMARY

Embodiments herein are directed to prebiotic cosmetic compositions. In various embodiments, such a cosmetic composition includes a pH buffer system, wherein the pH buffer system includes an organic acid or an organic base; from about 0.1 to about 10 weight percent of a prebiotic based on a total weight of the cosmetic composition, wherein the prebiotic includes at least one carbohydrate compound; and from about 0.1 to about 5 weight percent of at least one fatty acid ester based on a total weight of the cosmetic composition, or from about 0.1 to about 2 weight percent of at least one biosurfactant based on a total weight of the cosmetic composition, wherein the cosmetic composition has a body area pH, and wherein at least one of the organic acid, the organic base, the prebiotic, the at least one fatty acid ester, and the at least one biosurfactant are bio-based compounds.

In certain embodiments, the cosmetic composition is a hygienic cosmetic composition that includes a non-pigmented composition, an undyed composition, a liquid soap, a face wash, a body wash, a mouthwash, a shampoo, a hair conditioner, a deodorant, a toothpaste, a moisturizer, a lip balm, a body lotion, a serum, a skin tonic, a shave cream, a bar soap, an anhydrous balm, or combinations thereof.

In certain embodiments, the cosmetic composition includes from about 1 to about 5 weight percent of the prebiotic based on the total weight of the cosmetic composition. In certain embodiments, the cosmetic composition includes from about 2 to about 4 weight percent prebiotic based on the total weight of the cosmetic composition. In various embodiments, the prebiotic is capable of selectively being utilized by a microorganism on a body area to provide a health benefit.

In certain embodiments, the cosmetic composition includes from about 0.2 to about 4 weight percent of at least one fatty acid ester based on the total weight of the cosmetic composition. In certain embodiments, the prebiotic and the at least one fatty acid ester have a combined weight percent of about 10 weight percent or less based on the total weight of the cosmetic composition. In certain embodiments, a ratio of the prebiotic to the at least one fatty acid ester is a weight ratio in a cosmetic composition is from about 3:1 to about 5:1.

In certain embodiments, the at least one fatty acid ester includes a monoglyceride, a polyglyceride, a medium-chain triglyceride, xylityl sesquicaprylate, propanediol caprylate, a fatty acid ester of a monohydric alcohol, octadecanoic acid monoester with a diol, octadecanoic acid monoester with 1,2-propanediol, octadecanoic acid monoester with glycerol, an undec-10-enoic acid monoester with a diol, undec-10-enoic acid monoester with propane-1,3-diol, undec-10-enoic acid monoester with glycerol, or a combination thereof, wherein the medium-chain triglyceride has an average chain length of from 6 carbon atoms to 12 carbon atoms. In certain embodiments, the at least one fatty acid ester inhibits a growth of a harmful or malodorous bacterial population or a fungus.

In certain embodiments, the at least one carbohydrate compound includes an inulin, an alpha-glucan oligosaccharide, a fructooligosaccharide, an isomaltooligosaccharide, a xylooligosaccharide, an arabinoxylo-oligosaccharide, a beta-glucan, a transgalactooligosaccharide, a mannan-oligosaccharide, lactulose, xylitol, lactitol, trehalose, or combinations thereof.

In certain embodiments, the at least one organic acid includes glycolic acid, citric acid, lactic acid, malic acid, tartaric acid, mandelic acid, salicylic acid, gluconic acid, acetic acid, succinic acid, phytic acid, galacturonic acid, anisic acid, levulinic acid, azelaic acid, shikimic acid, usnic acid, or a combination thereof. In certain embodiments, the at least one organic base includes sodium bicarbonate, calcium carbonate, or a combination thereof.

In certain embodiments, a body area pH is about 5.5 to about 6.5 for an under arm skin, about 4.5 to about 5.5 for a scalp skin, about 3.8 to about 4.5 for a vaginal skin, about 6.7 to about 7.3 for an oral cavity skin, or about 4.0 to about 7.0 for a body skin.

In certain embodiments, the cosmetic composition includes an aqueous carrier. In certain embodiments, the cosmetic composition is anhydrous.

In certain embodiments, the cosmetic composition includes from about 0.1 to about 5 weight percent of at least one fatty acid ester and from about 0.1 to about 2 weight percent of at least one biosurfactant based on the total weight of the cosmetic composition. In certain embodiments, the biosurfactant includes an alkyl glyceryl ether. In certain embodiments, the alkyl glyceryl ether includes caprylyl glyceryl ether. In certain embodiments, the biosurfactant includes a rhamnolipid, a sophorolipid, surfactin, and combinations thereof.

In certain embodiments of a cosmetic composition herein, at least two of the organic acid, the organic base, the prebiotic, the at least one fatty acid ester, and the at least one biosurfactant are bio-based compounds. In other embodiments, at least three of the organic acid, the organic base, the prebiotic, the at least one fatty acid ester, and the at least one biosurfactant are bio-based compounds. In some embodiments, the cosmetic composition excludes from about 5 weight percent or more of one or more of a non-natural component based on the total weight of the cosmetic composition. In certain embodiments, the cosmetic composition excludes from 1 weight percent or more of the non-natural component based on the total weight of the cosmetic composition. In certain embodiments, the non-natural component includes a paraben, an aluminum compound, a broad-spectrum antibiotic, a formaldehyde containing compound, a phthalate, a heavy metal, benzyl alcohol, alkyl benzene sulfonates, ammonium lauryl sulfate, sodium lauryl sulfate, tetrasodium ethylenediaminetetraacetic acid, or combinations thereof.

Various embodiments herein provide a kit for adding prebiotics to a cosmetic product including at least one prebiotic vessel containing a prebiotic composition, wherein the prebiotic composition includes at least one carbohydrate compound; and at least one antimicrobial vessel containing an antimicrobial composition, wherein the antimicrobial composition includes at least one fatty acid ester, or at least one biosurfactant. In other embodiments, a kit includes at least one combined vessel containing a combined composition, wherein the combined composition includes the antimicrobial composition and the prebiotic, wherein the prebiotic includes at least one carbohydrate compound and the antimicrobial composition includes the at least one fatty acid ester, or at least one biosurfactant.

In some embodiments of a kit herein, the prebiotic composition includes from about 0.5 to about 50 percent weight of the prebiotic based on a total weight of the prebiotic composition. In certain embodiments, the antimicrobial composition includes from about 0.5 to about 25 weight percent of the at least one fatty acid ester or the at least one biosurfactant, based on a total weight of the antimicrobial composition. In certain embodiments, the combined composition includes from about 1 to about 75 weight percent of the prebiotic and the antimicrobial composition based on a total weight of the combined composition.

Methods of adding prebiotics to a cosmetic product are disclosed herein. Embodied methods of adding prebiotics to a cosmetic product herein include providing a kit for adding a prebiotic liquid to a cosmetic product, wherein the kit includes at least one prebiotic vessel containing a prebiotic composition, wherein the prebiotic composition includes at least one prebiotic, and the at least one prebiotic includes at least one carbohydrate compound; and at least one antimicrobial vessel containing an antimicrobial composition that includes at least one fatty acid ester or at least one biosurfactant; and adding the prebiotic composition and the antimicrobial composition to the cosmetic product to form the prebiotic cosmetic product. In certain embodiments, the prebiotic composition and the antimicrobial composition are added to the cosmetic product in a weight ratio of from about 3:1 to about 5:1.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one of the organic acid, the organic base, the prebiotic, the at least one fatty acid ester, and the at least one biosurfactant” means an organic acid, an organic base, a prebiotic, one or more fatty acid esters, or one or more biosurfactants, or any combination thereof.

Unless otherwise noted, the term “about” refers to ±10% of the non-percentage number that is described, rounded to the nearest whole integer. For example, about 100 nm, would include from 90 nm to 110 nm. Unless otherwise noted, the term “about” refers to ±5% of a percentage number. For example, about 10% would include 5% to 15%. When the term “about” is discussed in terms of a range, then the term refers to the appropriate amount less than the lower limit and more than the upper limit. For example, from about 10 to about 100 would include from 9 nm to 110 nm. An exception is when the term “about” is used for pH because pH has a logarithmic scale. For pH measurements, a pH of about 7 means ±0.2 pH so a pH of about 7.0 would include 6.8 and 7.2.

Unless otherwise noted, the term “prebiotic” refers to a compound that can be utilized by host microorganisms to confer a health benefit.

Unless otherwise noted, the term “microbiome” refers to the collective genomes of the microbes (composed of bacteria, bacteriophage, fungi, protozoa and viruses) that live inside and on the human body.

Unless otherwise noted, the term “body area” refers to an area of the body's integumentary system, including but not limited to the skin, scalp, exocrine glands, axillary areas, mucous membranes of the mouth oral cavity, vagina, and anus; hair, nails, teeth, hooves, feathers, fur, scales, and claws.

Unless otherwise noted, the term “bio-based” refers to a compound or substance that is produced or has been produced by a living organism in nature, whether being extracted directly from a natural source, or that is synthetically produced to have the equivalent molecular structure or configuration as the compound or substance as it exists in nature. In various embodiments herein, a “bio-based” refers to a compound or substance, wherein from 30 to 100 weight percent of the compound, based on a total weight of the bio-based compound or substance, is made from carbon-based, renewable, biological resources. By counter example, a compound containing more than 30 weight percent of a compound produced from petroleum products would not be “bio-based.” A “natural ingredient” refers to an ingredient wherein from 50 to 100 weight percent, based on a total weight of the natural ingredient, has a molecular structure that is unchanged relative to its original natural source.

Unless otherwise noted, the term “non-natural component” to an ingredient wherein from 50 to 100 weight percent, based on a total weight of the non-natural component, has a molecular structure that is not found in nature.

Unless otherwise noted, the term “non-toxic” means free from toxic materials prohibited by FDA regulations, including bithionol, chlorofluorocarbon propellants, chloroform, hexachlorophene, mercury compounds, methylene chloride, bovine spongiform encephalopathy, vinyl chloride, and zirconium-containing complexes as well as any color additive that is subject to or prohibited by FDA regulation for cosmetic products. By counter example, color additives approved by the FDA that are exempt from batch certification would be considered non-toxic.

Unless otherwise noted, the term “anhydrous” refers to containing 1 weight percent or less of water based on a total weight of a composition.

The term “antimicrobial composition” as used herein refers to compositions containing fatty acid esters and/or bio-surfactants, which have selective, narrow antimicrobial properties against harmful bacteria and fungi.

The body's integumentary system, with all of its varied types of surfaces and environments, is host to hundreds of microbial species, including bacteria, bacteriophage, fungi, protozoa and viruses. The makeup of the microbial community varies with different body areas, depending on temperature, moisture levels, pH, and sebum levels. The microorganisms living inside and on the body are collectively termed the “microbiota”, or “microbiome” when referring to their collective genomes. Many of these commensal microorganisms are not harmful, coexisting without damaging the host, while others are beneficial, providing health benefits. Such symbiotic microorganisms can help to protect the body against infection and overgrowth by harmful or pathogenic microorganisms. The skin microbiota exists together with the host as an ecosystem in delicate balance with the environment. A healthy balance can provide an effective barrier against harmful microbes and toxic substances, and can act to guard against inflammation and to promote would healing. Disruption of this balance, called dysbiosis, can result in disorders and infections, including many common skin ailments.

Unfortunately, modern hygienic practices can be destructive to the balance of microbiota in various integumentary body areas. In recent decades, the commensal microbiome has been negatively impacted not only by poor dietary habits, but by the frequent and at times excessive use of personal care products, disinfectants, antibiotics, and other germ killers.

The use of personal care products such as cleansers, conditioners, deodorants, balms and lotions can upset the balance of the body's microbiota. For instance, imbalances in the skin microbes can occur that lead to alterations of the skin barrier. The personal care industry has designed many hygienic cosmetic products to eradicate skin bacteria or other microorganisms, targeting the spread of germs or the treatment of skin disorders. Such products, however, can kill off the commensal “good” microflora along with the “bad”, not taking into account the importance of preserving the beneficial balance of the skin microbiota. Antibacterial ingredients in soaps, preservatives in topical products, and other harsh chemicals applied to the skin, can kill the beneficial bacteria as well as the harmful ones. Deodorants were among the first products developed to target skin microorganisms, following early studies on the effect of topical antibacterial agents on the axillary areas. Antibacterial ingredients have been widely used in deodorants; but because of major safety concerns, some of these agents, such as farnesol and triclosan, have gradually been restricted or banned in most countries.

An imbalance of the skin microbiota can lead to a variety of common skin conditions, including acne, eczema, rosacea, and even premature aging. The incidence of such skin conditions has risen dramatically along with the cultural emphasis on cleanliness. The incidence of eczema, or atopic dermatitis, has increased two to three-fold in industrialized nations, impacting approximately 15% to 20% of children and 1% to 3% of adults worldwide. Clinical research has shown that compared to healthy skin, blemish-prone skin has a less diverse skin microbiome and overpopulations of pathogens. A balanced skin microbiome, however, can be effective to prevent or reduce the severity of such conditions.

Embodiments of cosmetic compositions herein can provide a benefit of promoting a healthy balance of microorganisms in a body area. Such embodiments can provide the advantages of encouraging the growth of commensal or symbiotic microflora, and inhibiting the ability of pathogenic and opportunistic microorganisms to grow or to invade the body area. Such embodiments can provide health benefits of promoting, restoring, reinforcing and maintaining the defense mechanisms provided by a balanced microbiota. Embodiments of cosmetic compositions herein can also provide a benefit of a safer approach to the modulation of body odor.

It may take some re-education, but the goal is to persuade consumers to embrace prebiotic cosmetics with the same fervor they did antibacterial ones. Those who make the switch will be heathier for doing so.

A benefit of embodiments herein can be providing bio-based materials in cosmetic compositions. The use of bio-based materials, including materials from sustainable sources, can be helpful to minimize impact on the environment. Such materials can include upcycled bio-based raw materials derived from byproducts of the food industry or obtained from bio-fermentation. Such materials can include compounds and substances that are produced naturally by a microbial population, derived directly from a microbial population, or synthesized according to the structure of a compound or substance as it naturally occurs in the microbial population. Such a microbial population can be a population that occurs in a body area microbiota.

There are many cosmetics on the market and consumers are bound to desire the benefits of a prebiotic cosmetic for use with their favorite, trusted cosmetic product. Embodiments herein can provide kits for adding a prebiotic liquid and an antimicrobial composition to a cosmetic product. Such embodiments can impart advantages of the cosmetic compositions disclosed herein to a conventional cosmetic product, provided that the conventional cosmetic product is not overburdened with broad spectrum antibiotics or non-natural components.

### Embodiments of Cosmetic Compositions

Embodiments of cosmetic compositions as disclosed herein can include a pH buffer system. In various embodiments, the pH buffer system includes an organic acid or an organic base. In various embodiments, the pH buffer system includes an organic acid and an organic base. In various embodiments, the cosmetic composition includes from about 0.1 to about 10 weight percent of a prebiotic based on a total weight of the cosmetic composition, the prebiotic including at least one carbohydrate compound. In some embodiments, the cosmetic composition contains from about 0.1 to about 5 weight percent of at least one fatty acid ester. In some embodiments, the cosmetic composition includes from about 0.1 to about 2 weight percent of at least one biosurfactant based on a total weight of the cosmetic composition. In some embodiments, the cosmetic composition includes from about 0.1 to about 5 weight percent of the at least one fatty acid ester, and from about 0.1 to about 2 weight percent of at least one biosurfactant based on the total weight of the cosmetic composition. In various embodiments, the cosmetic composition has a body area pH. In various embodiments, at least one of the organic acid, the organic base, the prebiotic, the at least one fatty acid ester, and the at least one biosurfactant are bio-based compounds. In some embodiments, the cosmetic composition includes an aqueous carrier. In some embodiments, the cosmetic composition is anhydrous.

In certain embodiments, the cosmetic composition is a hygienic cosmetic composition. In some embodiments, the hygienic cosmetic composition includes a non-pigmented composition, an undyed composition, a liquid soap, a face wash, a body wash, a mouthwash, a shampoo, a hair conditioner, a deodorant, a toothpaste, a moisturizer, a lip balm, a body lotion, a serum, a skin tonic, a shave cream, a bar soap, an anhydrous balm, a foam, a gel, an emollient, a hair oil, a cuticle cream, an ointment, or combinations thereof.

In some embodiments of a cosmetic composition, at least two of the organic acid, the organic base, the prebiotic, the at least one fatty acid ester, and the at least one biosurfactant are bio-based compounds. In some embodiments, at least three of the organic acid, the organic base, the prebiotic, the at least one fatty acid ester, and the at least one biosurfactant are bio-based compounds.

In some embodiments, the cosmetic composition excludes from about 0.05 weight percent or more of one or more of non-natural components based on the total weight of the cosmetic composition, including from about 5 weight percent to about 50 weight percent non-natural component. In some embodiments, the cosmetic composition excludes from 0.01 weight percent or more, including from 1 weight percent to about 50 weight percent, of the non-natural component based on the total weight of the cosmetic composition. In some embodiments, the non-natural component includes a paraben, an aluminum compound, a broad-spectrum antibiotic, a formaldehyde containing compound, a phthalate, a heavy metal, benzyl alcohol, alkyl benzene sulfonates, ammonium lauryl sulfate, sodium lauryl sulfate, tetrasodium ethylenediaminetetraacetic acid (EDTA), or combinations thereof. In some embodiments, the non-natural component includes a non-natural acid, including but not limited to an inorganic acid. In some embodiments, the non-natural component includes a non-natural prebiotic, a non-natural fatty acid ester, and a non-natural biosurfactant. In some embodiments, the non-natural component includes a non-natural antibiotic, including but not limited to triclosan and farnesol. In some embodiments, the non-natural component includes but is not limited to a formaldehyde releasing compound, lead, manganese, titanium, nickel, cobalt, cadmium, zinc oxide, titanium dioxide, and salicylic acid. If the amount of non-natural component excluded goes below the lower limit of 0.01 weight percent, then the harmful effects may no longer overburden the cosmetic compositions.

Embodiments of cosmetic compositions herein can provide a benefit of a pH buffer system, a prebiotic, and a fatty acid ester or a biosurfactant, all having properties that can work together in the composition to promote, restore and maintain a healthy balance of microorganisms in an integumentary body area. Such properties can include the stimulation of growth of commensal or symbiotic microorganisms in the body area, and the inhibition of growth or invasion of opportunistic, harmful or pathogenic microorganisms in the body area. A harmful bacterial population can include a bacterial population that can cause a common skin condition, including but not limited to acne, eczema, rosacea, dermatitis, and common skin infections. A benefit of embodiments of cosmetic compositions herein can include one or more components in the pH buffer system, prebiotic, fatty acid ester or biosurfactant that are bio-based components that are naturally produced by a microbial population that occurs in a body area microbiota. Such a composition can mimic the natural defenses that microbiota populations can provide to a body area, and can thus enhance the body area's innate resistance to harmful microbial colonization, growth and infection.

### pH Buffer Systems of Various Embodiments

Various embodiments of a cosmetic composition herein include a pH buffer system. In various embodiments, the pH buffer system can include an organic acid or an organic base. In various embodiments, the pH buffer system maintains the cosmetic composition at a body area pH. In certain embodiments, the body area pH is about 5.5 to about 6.5 for an underarm skin. In some embodiments, the body area pH for an underarm skin is about 5.7 to about 6.3; in some embodiments, the body area pH for an underarm skin is about 5.9 to about 6.1. In certain embodiments, the body area pH is about 4.5 to about 5.5 for a scalp skin. In some embodiments, the body area pH for a scalp skin is about 4.7 to about 5.3. In some embodiments, the body area pH for a scalp skin is about 4.9 to about 5.1. In certain embodiments, the body area pH is about 3.8 to about 4.5 for a vaginal skin. In some embodiments, the body area pH for a vaginal skin is about 4.0 to about 4.3. In some embodiments, the body area pH for a vaginal skin is about 4.1 to about 4.2. In certain embodiments, the body area pH is about 6.7 to about 7.3 for an oral cavity skin. In some embodiments, the body area pH for an oral cavity skin is about 6.9 to about 7.1. In certain embodiments, the body area pH is about 4.0 to about 7.0 for a body skin. In some embodiments, the body area pH for a body skin is about 4.5 to about 6.5. In some embodiments, the body area pH for a body skin is about 5.0 to about 6.0. In some embodiments, the body area pH for a body skin is about 5.3 to about 5.7. The term “body skin” refers to the general parts of the skin that are not included in the aforementioned body areas, such as skin on the arm, leg, etc.

In various embodiments, the at least one organic acid of the pH buffer system can include glycolic acid, citric acid, lactic acid, malic acid, tartaric acid, mandelic acid, salicylic acid, gluconic acid, acetic acid, succinic acid, phytic acid, galacturonic acid, anisic acid, levulinic acid, azelaic acid, shikimic acid, usnic acid, or a combination thereof. An organic acid according to embodiments herein can be an organic acid that is produced by, derived from, or synthesized according to the structure of an organic acid naturally occurring in a microbial or a bacterial population. Such a microbial or bacterial population can include a population that naturally occurs in a body area microbiota. An organic acid can provide a benefit of having an antimicrobial property. In various embodiments, the at least one organic base of the pH buffer system can include sodium bicarbonate, calcium carbonate, or a combination thereof.

### Prebiotics of Various Embodiments

Various embodiments of a cosmetic composition herein include from about 0.1 to about 10 weight percent of a prebiotic based on a total weight of the cosmetic composition. In some embodiments, the cosmetic composition includes from about 1 to about 8 weight percent of the prebiotic based on a total weight of the cosmetic composition. In some embodiments, the cosmetic composition includes from about 2 to about 4 weight percent prebiotic based on a total weight of the cosmetic composition. If the concentration of prebiotic goes below 0.1 weight percent, then the beneficial effects will be lost. If the concentration of prebiotic passes above 10 weight percent, then the cosmetic composition will become unacceptable sticky to consumers and they will not buy or use it, which limits the health benefits of the cosmetic composition.

In various embodiments, the prebiotic includes at least one carbohydrate compound. Such a carbohydrate compound can be a bio-based compound. Such a carbohydrate compound can be plant derived. A benefit of a carbohydrate compound can be improving the balance of a body area commensal microbiota, by providing an energy source to promote beneficial microbial growth in the body area. In some embodiments, the at least one carbohydrate compound includes an inulin, an alpha-glucan oligosaccharide, a fructooligosaccharide, an isomaltooligosaccharide, an xylooligosaccharide, an arabinoxylo-oligosaccharide, a beta-glucan, a transgalactooligosaccharide, mannan-oligosaccharide, lactulose, xylitol, lactitol, trehalose, or combinations thereof. In an embodiment of a cosmetic composition, a deodorant formula can include an inulin or a polyfructose carbohydrate. Such an inulin or polyfructose carbohydrate can be extracted from chicory roots.

In various embodiments, the prebiotic is capable of being selectively utilized by a microorganism on a body area to provide a health benefit. Such a prebiotic can provide a health benefit of selective stimulation of beneficial commensal and symbiotic microorganisms, competitively against and to the detriment of the growth of opportunistic, undesirable, or pathogenic microorganisms. Such a health benefit can include helping commensal and beneficial microorganisms to flourish in a body area. A health benefit can include protecting and promoting the balance and diversity of the microbiota in a body area. Another health benefit can include increasing a body area's defense against excessive growth or infection by harmful microbes, or against external toxic substances. Such a health benefit can include the stimulation of antimicrobial peptide release by keratinocytes in the skin. Another health benefit can include increasing the skin's resistance to pathological conditions, including but not limited to dermatitis, acne, rosacea, eczema, and premature aging.

### Fatty Acid Esters and Biosurfactants of Various Embodiments

Certain embodiments of a cosmetic composition herein include from about 0.1 to about 5 weight percent of at least one fatty acid ester based on a total weight of the cosmetic composition. In some embodiments, the cosmetic composition includes from about 0.2 to about 4 weight percent of at least one fatty acid ester based on a total weight of the cosmetic composition. In some embodiments, the cosmetic composition includes from about 0.5 to about 3 weight percent of at least one fatty acid ester based on a total weight of the cosmetic composition. If the concentration of fatty acid ester goes below 0.1 weight percent, then the beneficial effects will be lost. If the concentration of fatty acid ester passes above 5 weight percent, then the cosmetic composition suffers from formula instability, an unpleasant texture, and/or potentially eye and skin irritation.

In certain embodiments, the cosmetic composition includes a prebiotic and at least one fatty acid ester in a combined weight percent of about 10 percent or less based on a total weight of the cosmetic composition. In some embodiments, the prebiotic and the at least one fatty acid ester have a combined weight percent of about 5 percent or less based on the total weight of the cosmetic composition. In some embodiments, a ratio of the prebiotic to the at least one fatty acid ester is a weight ratio from about 3:1 to about 5:1.

In various embodiments of a cosmetic composition, the at least one fatty acid ester can include a monoglyceride, a polyglyceride, a medium-chain triglyceride, xylityl sesquicaprylate, propanediol caprylate, a fatty acid ester of a monohydric alcohol, octadecanoic acid monoester with a diol, octadecanoic acid monoester with 1,2-propanediol, octadecanoic acid monoester with glycerol, an undec-10-enoic acid monoester with a diol, undec-10-enoic acid monoester with propane-1,3-diol, undec-10-enoic acid monoester with glycerol, or a combination thereof. In some embodiments, the medium-chain triglyceride has an average chain length of from 6 carbon atoms to 12 carbon atoms. A fatty acid ester according to embodiments herein can be a bio-based compound. A fatty acid ester according to embodiments herein can have a structure based on one or more free fatty acids naturally found on the skin surface.

In various embodiments, the at least one fatty acid ester inhibits a growth of a harmful or malodorous bacterial population. In some embodiments, the fatty acid ester inhibits a growth of a fungus. A harmful bacterial population can include a bacterial population that can cause a common skin condition, including but not limited to acne, eczema, rosacea, dermatitis, and common skin infections. A malodorous bacterial population can include a bacterial population that can cause an unpleasant body odor, including but not limited to axillary body odors and bad breath.

Various embodiments of a cosmetic composition herein can include from about 0.1 to about 2 weight percent of at least one biosurfactant based on a total weight of the cosmetic composition. If the concentration of at least one biosurfactant goes below 0.1 weight percent, then the beneficial effects will be lost. If the concentration of at least one biosurfactant passes above 5 weight percent, then the cosmetic composition suffers from formula instability, an unpleasant texture, and/or potentially eye and skin irritation

In some embodiments, the cosmetic composition can include from about 0.5 to about 1 weight percent of at least one biosurfactant based on a total weight of the cosmetic composition. In some embodiments, the cosmetic composition includes from about 0.1 to about 5 weight percent of at least one fatty acid ester, and from about 0.1 to about 2 weight percent of at least one biosurfactant based on a total weight of the cosmetic composition. A biosurfactant according to embodiments herein can provide a benefit of reducing surface tension of liquids and increasing solubility of materials in a body area, thereby reducing the adherence of microorganisms on the surface of the body area. In some embodiments, the at least one biosurfactant includes an alkyl glyceryl ether. In some embodiments, the alkyl glyceryl ether includes caprylyl glyceryl ether. In some embodiments, the at least one biosurfactant includes a rhamnolipid, a sophorolipid, surfactin, and combinations thereof.

### Kits of Various Embodiments

Embodiments herein provide a kit for adding prebiotics to a cosmetic product. In some embodiments, the kit includes at least one prebiotic vessel containing a prebiotic composition, wherein the prebiotic composition includes at least one carbohydrate compound; and at least one antimicrobial vessel containing an antimicrobial composition, wherein the antimicrobial composition includes at least one fatty acid ester or at least one biosurfactant. In some embodiments, the kit includes at least one combined vessel containing a combined composition, wherein the combined composition includes an antimicrobial composition and a prebiotic, wherein the prebiotic includes at least one carbohydrate compound and the antimicrobial composition includes at least one fatty acid ester or at least one biosurfactant.

In some embodiments of a kit herein, the prebiotic composition can include from about 0.5 to about 50 percent weight of a prebiotic based on a total weight of the prebiotic composition. In some embodiments, the prebiotic composition can include from about 1 to about 40 percent of a prebiotic based on a total weight of the prebiotic composition. In some embodiments, the prebiotic composition can include from about 10 to about 30 percent of a prebiotic based on a total weight of the prebiotic composition.

In some embodiments, the antimicrobial composition can include from about 0.5 to about 25 weight percent of at least one fatty acid ester based on a total weight of the antimicrobial composition. In some embodiments, the antimicrobial composition can include from about 1 to about 20 percent weight of at least one fatty acid ester based on a total weight of the antimicrobial composition. In some embodiments, the antimicrobial composition can include from about 5 to about 10 percent weight of at least one fatty acid ester based on a total weight of the antimicrobial composition.

In some embodiments, the antimicrobial composition can include at least one biosurfactant based on a total weight of the antimicrobial composition. In some embodiments, the antimicrobial composition can include from about 0.5 to about 25 weight percent of at least one biosurfactant based on a total weight of the antimicrobial composition. In some embodiments, the antimicrobial composition can include from about 1 to about 20 weight percent of at least one biosurfactant based on a total weight of the antimicrobial composition.

In some embodiments the antimicrobial composition can include from about 0.5 to about 25 weight percent of at least one fatty acid ester based on a total weight of the antimicrobial composition, and from about 0.5 to about 25 weight percent of at least one biosurfactant based on a total weight of the antimicrobial composition.

In some embodiments, the combined composition includes from about 1 to about 75 weight percent of the prebiotic and the antimicrobial composition based on a total weight of the combined composition. In some embodiments, the combined composition includes from about 10 to about 60 weight percent of the prebiotic and the antimicrobial composition based on a total weight of the combined composition. In some embodiments, the combined composition includes from about 20 to about 50 weight percent of the prebiotic and the antimicrobial composition based on a total weight of the combined composition.

### Embodiments of Methods of Adding Prebiotics to a Cosmetic Product

Embodiments herein provide methods of adding prebiotics to a cosmetic product. In some embodiments, a method provides a kit for adding a prebiotic liquid to a cosmetic product. In certain embodiments, the kit includes at least one prebiotic vessel containing a prebiotic composition, wherein the prebiotic composition includes at least one prebiotic, and the at least one prebiotic includes at least one carbohydrate compound; and at least one antimicrobial vessel containing an antimicrobial composition that includes at least one fatty acid ester or at least one biosurfactant. In some embodiments, the antimicrobial composition can include at least one fatty acid ester and at least one biosurfactant. Various embodiments of the methods include adding the prebiotic composition and the antimicrobial composition to a cosmetic product to form a prebiotic cosmetic product. In some embodiments, the prebiotic composition and the antimicrobial composition are added to the cosmetic product in a weight ratio of from about 3:1 to about 5:1.

### Examples

A body wash is prepared containing 5 weight percent of xylitol based on the composition total weight, and 1 weight percent of xylityl sesquicaprylate based on the composition total weight. The composition is buffered to pH 7.0 using citric acid.

What is claimed is:

1. A cosmetic composition comprising:
a pH buffer system, wherein the pH buffer system includes an organic acid or an organic base;
from about 0.1 to about 10 weight percent of a prebiotic based on a total weight of the cosmetic composition, wherein the prebiotic includes at least one carbohydrate compound; and
from about 0.1 to about 5 weight percent of at least one fatty acid ester or from about 0.1 to about 2 weight percent of at least one biosurfactant based on a total weight of the cosmetic composition,
wherein the cosmetic composition has a body area pH, and
wherein at least one of the organic acid, the organic base, the prebiotic, the at least one fatty acid ester, and the at least one biosurfactant are bio-based compounds.

2. The cosmetic composition of claim 1, wherein the at least one fatty acid ester inhibits a growth of a harmful or malodorous bacterial population or a fungus.

3. The cosmetic composition of claim 1, wherein the cosmetic composition is a hygienic cosmetic composition that includes a non-pigmented composition, an undyed composition, a liquid soap, a face wash, a body wash, a mouthwash, a shampoo, a hair conditioner, a deodorant, a toothpaste, a moisturizer, a lip balm, a body lotion, a serum, a skin tonic, a shave cream, a bar soap, an anhydrous balm, or combinations thereof.

4. The cosmetic composition of claim 1, wherein the body area pH is about 5.5 to about 6.5 for an under arm skin, about 4.5 to about 5.5 for a scalp skin, about 3.8 to about 4.5 for a vaginal skin, about 6.7 to about 7.3 for an oral cavity skin, or about 4.0 to about 7.0 for a body skin.

5. The cosmetic composition of claim 1, wherein the cosmetic composition includes from about 1 to about 5 weight percent of the prebiotic based on the total weight of the cosmetic composition, or
from about 2 to about 4 weight percent prebiotic based on the total weight of the cosmetic composition.

6. The cosmetic composition of claim 1, wherein the cosmetic composition includes from about 0.2 to about 4 weight percent of the at least one fatty acid ester based on the total weight of the cosmetic composition.

7. The cosmetic composition of claim 1, wherein the prebiotic and the at least one fatty acid ester have a combined weight percent of about 10 weight percent or less based on the total weight of the cosmetic composition; or
wherein a ratio of the prebiotic to the at least one fatty acid ester is a weight ratio from about 3:1 to about 5:1.

8. The cosmetic composition of claim 1, wherein the at least one organic acid includes glycolic acid, citric acid, lactic acid, malic acid, tartaric acid, mandelic acid, salicylic acid, gluconic acid, acetic acid, succinic acid, phytic acid, galacturonic acid, anisic acid, levulinic acid, azelaic acid, shikimic acid, usnic acid, or a combination thereof; or the at least one organic base includes sodium bicarbonate, calcium carbonate, or a combination thereof.

9. The cosmetic composition of claim 1, wherein the at least one carbohydrate compound includes an inulin, an alpha-glucan oligosaccharide, a fructooligosaccharide, an isomaltooligosaccharide, a xylooligosaccharide, an arabinoxylo-oligosaccharide, a beta-glucan, a transgalactooligosaccharide, a mannan-oligosaccharide, lactulose, xylitol, lactitol, trehalose, or combinations thereof.

10. The cosmetic composition of claim 1, wherein the prebiotic is capable of selectively being utilized by a microorganism on a body area to provide a health benefit, or
wherein the cosmetic composition further comprises an aqueous carrier, or
wherein the cosmetic composition is anhydrous.

11. The cosmetic composition of claim 1, wherein the at least one fatty acid ester includes a monoglyceride, a polyglyceride, a medium-chain triglyceride, xylityl sesquicaprylate, or a combination thereof,
wherein the medium-chain triglyceride has an average chain length of from 6 carbon atoms to 12 carbon atoms.

12. The cosmetic composition of claim 1, wherein the cosmetic composition includes from about 0.1 to about 5 weight percent of at the least one fatty acid ester and from about 0.1 to about 2 weight percent of the at least one biosurfactant based on the total weight of the cosmetic composition.

13. The cosmetic composition of claim 1, wherein the at least one biosurfactant includes an alkyl glyceryl ether.

14. The cosmetic composition of claim 13, wherein the alkyl glyceryl ether includes caprylyl glyceryl ether, or
the at least one biosurfactant includes a rhamnolipid, a sophorolipid, surfactin, and combinations thereof.

15. The cosmetic composition of claim 1, wherein at least two of the organic acid, the organic base, the prebiotic, the at least one fatty acid ester, and the at least one biosurfactant are bio-based compounds; or
wherein at least three of the organic acid, the organic base, the prebiotic, the at least one fatty acid ester, and the at least one biosurfactant are bio-based compounds.

16. The cosmetic composition of claim 1, wherein the cosmetic composition excludes from about 5 weight percent or more of one or more of a non-natural component based on the total weight of the cosmetic composition, or
the cosmetic composition excludes from 1 weight percent or more of the non-natural component based on the total weight of the cosmetic composition.

17. The cosmetic composition of claim 16, wherein the non-natural component includes a paraben, an aluminum compound, a broad-spectrum antibiotic, a formaldehyde containing compound, a phthalate, a heavy metal, benzyl alcohol, alkyl benzene sulfonates, ammonium lauryl sulfate, sodium lauryl sulfate, tetrasodium ethylenediaminetetraacetic acid, or combinations thereof.

18-19. (canceled)

20. A method of adding prebiotics to a cosmetic product comprising:
providing a kit for adding a prebiotic liquid to a cosmetic product,
wherein the kit includes at least one prebiotic vessel containing a prebiotic composition, wherein the prebiotic composition includes at least one prebiotic, and the at least one prebiotic includes at least one carbohydrate compound, and
at least one antimicrobial vessel containing an antimicrobial composition that includes at least one fatty acid ester or at least one biosurfactant; and
adding the prebiotic composition and the antimicrobial composition to the cosmetic product to form the prebiotic cosmetic product.

21. The method of claim 20, wherein the prebiotic composition and the antimicrobial composition are added to the cosmetic product in a weight ratio of from about 3:1 to about 5:1.

22. The cosmetic composition of claim 1, wherein the at least one fatty acid ester includes a monoglyceride, a polyglyceride, a medium-chain triglyceride, xylityl sesquicaprylate, propanediol caprylate, a fatty acid ester of a monohydric alcohol, octadecanoic acid monoester with a diol, octadecanoic acid monoester with 1,2-propanediol, octadecanoic acid monoester with glycerol, an undec-10-enoic acid monoester with a diol, undec-10-enoic acid monoester with propane-1,3-diol, undec-10-enoic acid monoester with glycerol, or a combination thereof,
wherein the medium-chain triglyceride has an average chain length of from 6 carbon atoms to 12 carbon atoms.
